(19) United States
(12) Patent Application Publication
(10) Pub. No.: US 2009/0142804 A1
(43) Pub. Date: Jun. 4, 2009

(54) Process For Preparing Serine-Rich Protein Employing Cysteine Synthase (CYSK) Gene

(75) Inventors: Sang Yup LEE, Daejeon (KR); Mee-Jung Han, Daejeon (KR)

Correspondence Address: SUGHRUE MION, PLLC, 2100 PENNSYLVANIA AVENUE, N.W., SUITE 800, WASHINGTON, DC, 20037, US

(73) Assignee: Korea Advanced Institute of Science and Technology, Yuseong-gu (KR)

(21) Appl. No.: 12/271,003

(22) Filed: Nov. 14, 2008

Related U.S. Application Data
(63) Continuation-in-part of application No. 10/662,517, filed on Sep. 16, 2003, now abandoned.

(30) Foreign Application Priority Data
Feb. 12, 2003 (KR) 10-2003-0008689

(51) Int. Cl. C12P 21/04; C12N 1/21

(52) U.S. Cl. 435/69.1; 435/252.3

(57) ABSTRACT

The present invention relates to a process for preparing a serine-rich foreign protein comprising culturing a bacterium containing the cysteine synthase (cysK) gene and a gene encoding the serine-rich foreign protein. The present invention comprises the steps of culturing a bacterium transformed with an expression vector containing a gene encoding a serine-rich foreign protein and an expression vector containing the cysK gene, or a bacterium transformed with an expression vector containing the cysK gene and a gene encoding a serine-rich foreign protein and isolating the foreign protein therefrom. The present invention is expected to be widely used to increase the production yield of a serine-rich foreign protein.

This is a Continuation-in-Part of U.S. Ser. No. 10/662,517 filed on Sep. 16, 2003, which claims priority from Korean patent application 10-2003-0008689 filed on Feb. 12, 2003, all of which are incorporated herein by reference.

The present invention relates to a process for preparing a serine-rich protein, which comprises culturing bacteria containing a cysteine synthase (cysK) gene and a serine-rich protein-encoding gene, and more particularly to a process for preparing a serine-rich protein, which comprises culturing bacteria containing a cysteine synthase (cysK) gene and a gene encoding a serine-rich protein which has a cysteine content equal to or lower than the average cysteine content of host cell proteins of the bacteria, and harvesting a serine-rich foreign protein from the culture.

### BACKGROUND ART

E. coli is a strain commonly used for synthesis and production of a foreign protein and has been applied in production of proteins, such as interferon, interleukin 2, colony-stimulating factors, growth hormones, insulin-like growth factors and human serum albumin, by recombinant technology. Also, for efficient production of a foreign protein in E. coli, plasmid vector expressing a foreign protein, proper culture conditions, inhibiting conditions of degradation of the prepared foreign, and the like are required and various systems have been developed to satisfy these requirements (Weickert et al., Curr. Opin. Biotechnol., 7:494-9, 1996).

However, there has been a problem in that when a method commonly used at present is used, it is hard to improve production yield of a foreign protein, because much time is required after induction of expression. Therefore, efforts have been made to overcome the problem but there has not been a report of a satisfactory result.

Therefore, there is a continuous need to develop a process for preparing a foreign protein by E. coli in a high yield.

Accordingly, the present inventors have conducted researches and studies to develop a method of producing a foreign protein by E. coli in a high yield. As a result, we have discovered that when the serine-rich foreign protein is prepared in E. coli, the production yield of a serine-rich foreign protein can be increased by coexpression of a gene encoding the serine-rich foreign protein and the cysteine synthase (cysK) gene derived from a bacterium thereby completing the present invention.

### SUMMARY OF THE INVENTION

Thus, it is an object of the present invention to provide a process for preparing a serine-rich foreign protein comprising culturing a bacterium containing the cysK gene and a gene encoding the serine-rich foreign protein.

It is a further object of the present invention to provide a bacterium simultaneously transformed with a recombinant vector including a gene encoding a serine-rich foreign protein and a recombinant vector including the cysK gene, and to provide a bacterium transformed with a recombinant vector including both the cysK gene and a gene encoding a serine-rich foreign protein.

It is another object of the present invention to provide a process for preparing a serine-rich foreign protein using a microorganism transformed with either the cysK gene or a recombinant vector including the cysK gene.

In accordance with the present invention, the above and other objects can be accomplished by the provision of a process for preparing a serine-rich foreign protein comprising culturing a bacterium containing the cysK gene and a gene encoding the serine-rich foreign protein.

In the present invention, the serine-rich protein preferably has a cysteine content equal to or lower than the average cysteine content of host cell proteins of the bacterium. According to the present invention, the bacterium may be transformed with a vector containing both the cysK gene and a gene encoding the serine-rich foreign protein. Alternatively, the bacterium may be transformed with a vector containing the cysK gene and a vector containing a gene encoding the serine-rich foreign protein.

Also, in accordance with another aspect of the present invention, there is provided a recombinant vector containing the cysK gene and a gene encoding a serine-rich protein which has a cysteine content equal to or lower than the average cysteine content of host cell proteins.

In accordance with yet another aspect of the present invention, there is provided a bacterium transformed with a vector containing the cysK gene and a vector containing a gene encoding a serine-rich protein which has a cysteine content equal to or lower than the average cysteine content of host cell proteins of the bacterium.

In accordance with yet another aspect of the present invention, there is provided the use of the cysK gene or a recombinant vector including the cysK gene in a method for preparing a foreign protein using a transformed microorganism.

In the present invention, the cysK gene is preferably derived from E. coli.

In addition, the serine-rich protein is preferably selected from the group consisting of, but not limited to, leptin, silk protein and sericin.

### BRIEF DESCRIPTION OF DRAWINGS

Further objects and advantages of the invention can be more fully understood from the following detailed description taken in conjunction with the accompanying drawings in which:

FIG. 1 is a graph showing expression levels of the GlyA and CysK proteins in cells before and after inducing expression of leptin protein using recombinant E. coli;

FIG. 2 is a gene map of the pAC104CysK plasmid;

FIG. 3 is a gene map of the pEDIL-12p40 plasmid;

FIG. 4a is a graph showing changes in cell density, dry cell weight and quantity of the foreign protein according to the culture time, when the recombinant E. coli BL21(DE3)(pEDOb5) that can produce leptin is cultured;

FIG. 4b is a graph showing changes in cell density, dry cell weight and quantity of foreign proteins according to the culture time, when the recombinant E. coli BL21(DE3)(pEDOb5)(pAC104CysK) that can produce leptin and coexpress the cysK gene is cultured;

FIG. 5a is a graph showing the amino acid composition of the E. coli proteins;

FIG. 5b is a graph showing the amino acid composition of leptin;

FIG. 5c is a graph showing the amino acid composition of the G-CSF;

FIG. 5d is a graph showing the amino acid composition of IL-12p40;

FIG. 6a is a graph showing changes in cell density, dry cell weight and quantity of foreign proteins according to the culture time, when the recombinant E. coli BL21(DE3)(pEDIL-12p40) that can produce IL-12p40 is cultured;

FIG. 6b is a graph showing changes in cell density, dry cell weight and quantity of foreign proteins according to the culture time, when the recombinant E. coli BL21(DE3)(pEDIL-12p40)(pAC104CysK) that can produce IL-12p40 and coexpress the cysK gene is cultured;

FIG. 7 is a gene map of pgly-cysK; and

FIG. 8 shows the results of SDS-PAGE analysis for the expression level of a silk protein. In FIG. 8, lane M: a marker showing the standard molecular weight of the protein; lane 1: shows results obtained by inducing the expression of the protein in a strain, transformed with the plasmids pSH32 and pACYC184, at an OD₆₀₀ of 0.4; lane 2: shows results obtained by inducing the expression of the protein in a strain, transformed with the plasmids pSH32 and pTet-glyVXY, at an OD₆₀₀ of 0.4; and lane 3: shows results obtained by inducing the expression of the protein in a strain, transformed with the plasmids pSH32 and pgly-cysK, at an OD₆₀₀ of 0.4.

### DETAILED DESCRIPTION OF THE INVENTION

The present invention will now be described in detail.

Firstly, the terms used herein are defined as follows.

The term “specific amino acid rich protein” as used herein refers to a protein containing a specific amino acid more than an average amino acid composition in E. coli (Koonin et al., in Escherichia coli and Salmonella: Cellular and Molecular Biology (eds. Neidhardt, F. C. et al.) American Society for Microbiology, Washington, D.C., 2203-17, 1996). As is known in the art, the average amino-acid composition of proteins in E. coli comprises 10.5% leucine, 9.6% alanine, and 5.6% serine which ranks eighth among the amino acid contents of the amino acid composition. Thus, although the serine-rich protein has been defined herein as a protein which has a serine content of more than 5.6% and ranks at least third among the amino acid contents of the amino acid composition, the serine-rich protein is preferably a protein which has a serine content of more than 10% and, at the same time, ranks first or second among the amino acid contents of the amino acid composition. As used herein, the phrase “serine-rich protein which has a cysteine content equal to or lower than the average cysteine content of host cell proteins” refers to a serine-rich protein which has a cysteine content equal to or lower than the average cysteine content of total protein in host cells and, at the same time, has a high serine content.

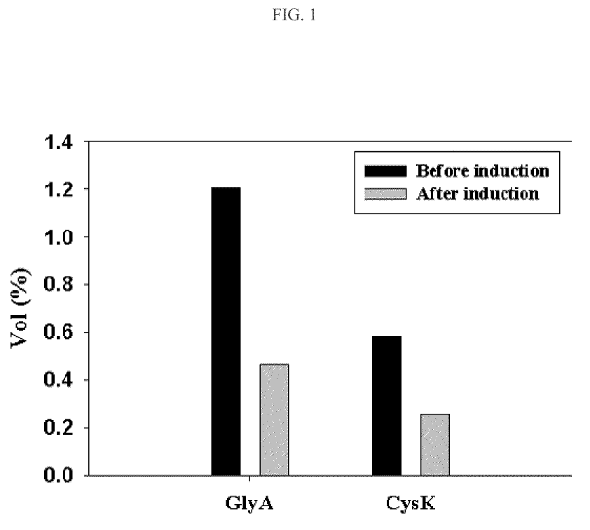

As known so far in the art, if a serine-rich protein such as leptin is produced in E. coli by DNA recombinant technology, leptin is prepared through the steps of culturing transformed E. coli at high concentration, inducing leptin expression, expressing leptin, harvesting transformed E. coli, extracting leptin from the E. coli., and the like. Here, a time of about more than 8 hours is required to reach to the highest leptin level after induction of the expression of leptin, and as analyzed in two-dimensional electrophoresis, the reason therefor is because the biosynthetic pathway of serine family amino acids in E. coli is inhibited while leptin-producing E. coli is cultured in high concentration (FIG. 1).

Silk protein is a serine-rich protein which consists of a repeat of a specific amino acid base sequence (SGRGGLGGTGAGMAA AAAMGGAGQGGYGGLGSQG) (SEQ ID NO: 1) and has a serine content as high as 6.3%. In one Example of the present invention, it was found that, when the cysK gene and the silk protein were coexpressed, the expression of the silk protein was further increased.

Thus, the present inventors have discovered that it is possible to reduce the production time of a serine-rich protein when coexpressing the gene encoding cysteine synthase that promotes synthesis of serine family amino acids along with a gene encoding a serine-rich protein, as compared to when expressing only a gene of a serine-rich protein, thereby increasing the production yield of the above-mentioned serine-rich proteins such as leptin and silk protein.

Meanwhile, leptin is a protein having a low cysteine content, and silk protein is a protein containing no cysteine. It was found in the present invention that the expression level of a serine-rich protein having a cysteine content equal to or lower than the average cysteine content of total protein in host cells was increased when a gene encoding cysteine synthase and the serine-rich protein were introduced and coexpressed in the host cells. Thus, it is considered that the reason why the expression level of the serine-rich protein is increased when the gene encoding cysteine synthase and the serine-rich protein are introduced and coexpressed in the host cells is not because the level of cysteine is increased due to the introduction of the cysteine synthase-encoding gene and that it is important to apply the serine-rich protein regardless the cysteine content thereof. Accordingly, in the present invention, the serine-rich protein is preferably a serine-rich protein which has a cysteine content equal to or lower than the average cysteine content of host cell proteins.

Accordingly, the present invention can be applied in order to increase the production yield of, in addition to the above-mentioned leptin and silk protein, sericin (SSTGSSSNTDSNSNSVGSSTSGGSSTYGYSSNSRDGSV) (SEQ ID NO: 2) and other synthetic proteins synthesized so as to have a cysteine content equal to or lower than the average cysteine content of host cell proteins.

### EXAMPLES

Now, the present invention will be described in detail by the following examples. However, it is apparent to those skilled in the art that the examples are only for illustrative purposes, and the present invention is not limited thereto.

### Example 1

### Assay of Physiological Change of Leptin Protein-Producing Strain Using Two-Dimensional Electrophoresis

The protein level changes before and after overproducing the human-derived leptin in recombinant E. coli BL21 (DE3)(pEDOb5) were compared by two-dimensional electrophoresis according to a known method (Hochstrasser et al., Anal. Biochem., 173:424-35, 1988; Han et al., J. Bacteriol., 183:301-8, 2001): Specifically, after initial culture of the E. coli BL21(DE3)(pEDOb5), the expression of leptin was induced, and the strain was cultured at high concentration. Culture broths were taken before and after the induction of expression. Each culture broth was centrifuged at 6000 rpm for 5 minutes at 4° C. to obtain precipitates, which were then washed with 500 [symbol] of low salt buffer (KCl 3 mM, KH₂PO₄ 1.5 mM, NaCl 68 mM, NaH₂PO₄ 9 mM). Then, the cells were suspended in 200 [symbol] of TE buffer (Tris-HCl 10 mM, EDTA 1 mM). The suspended cells were disrupted with a sonicator and centrifuged at 12,000 rpm for 10 minutes at 4° C. The supernatant was collected, dried in vacuo and stored at −20° C., which was used as a sample for the subsequent test.

200 μg of the prepared sample was dissolved in 340 [symbol] of modified IEF solution (urea 9M, CHAPS 0.5% (w/v), DTT 10 mM, Bio-lyte pH 3-10 0.2% (w/v), bromophenol blue 0.001% (w/v)) and applied to a 17 cm strip (ReadyStrip™ IPG Strips pH 3-10, Bio-Rad Laboratories Inc., USA). The strip was hydrated for 12 hours at 20° C. and subjected to isoelectric focusing. Then, the strip was dipped in equilibrated buffer I (urea 6M, SDS 2% (w/v), Tris-HCl (pH 8.8) 0.375M, glycerol 20% (v/v), DTT 130 mM) for about 15 minutes while shaking, and then was dipped in equilibrated buffer II (urea 6M, SDS 2% (w/v), Tris-HCl (pH 8.8) 0.375M, glycerol 20% (v/v), iodoacetamide 135 mM, bromophenol blue 3.5M) for about 15 minutes while shaking. The strip was loaded on SDS gel to carry out separation by molecular weight.

The two-dimensional gel was stained with a silver staining kit (Amersham Biosciences, Uppsala, Sweden), scanned with a scanner (GS710 Calibrated Imaging Densitometer, Bio-Rad Laboratories Inc., USA) and subjected to a quantitative analysis of protein by Melanie II software (Bio-Rad Laboratories Inc., USA). Also, for protein identification, desired proteins were taken selectively from the two-dimensional gel, washed, dried in vacuo and allowed to react with trypsin for 8 hours or more at 37° C. Then, the peptides cut by trypsin were measured for their molecular weights using MALDI-TOF MS (Matrix Assisted Laser Desorption/Ionization Time of Flight mass spectrometer) (Voyager™ Biospectrometry, Perseptive Biosystems Inc., USA). The protein levels before leptin expression were compared with those of the maximum content of leptin.

As a result, it was shown that synthesis of substantially all of the amino acids was inhibited after leptin gene expression. Particularly, the levels of enzymes involved in the synthesis of serine family amino acids (CysK, GlyA) were considerably reduced by excessive production of leptin protein, in which GlyA was reduced by 2.5 times and CysK was reduced by 2.3 times (FIG. 1). From this fact, it was noted that the biosynthesis of serine family amino acids was considerably impeded by the excessive production of leptin, and thus, in order to promote metabolism related to reduced biosynthesis of serine family amino acids in the strain producing leptin, the serine-rich protein, the cysK gene encoding the CysK protein which is a critical enzyme in this pathway was to be introduced.

### Example 2

### Preparation of Recombinant Plasmid with cysK Gene Introduced

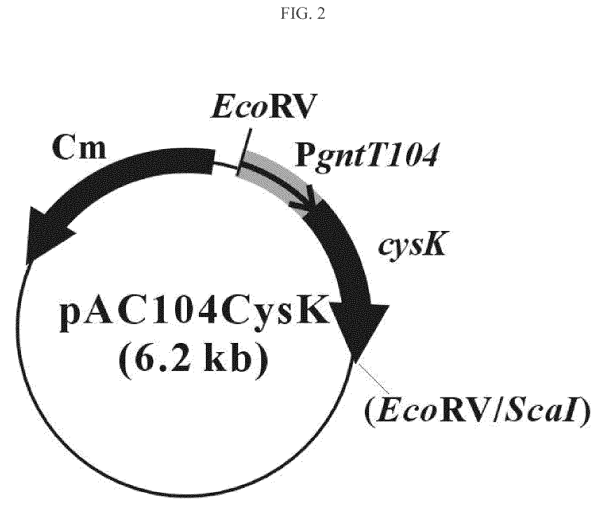

The recombinant plasmid pAC104CysK to express the CysK protein was prepared as follows: Firstly, polymerase chain reaction (PCR) was conducted using the E. coli BL21(DE3) chromosome as a template, and primer 1: 5′-gcgaattcatgagtaagatttttgaagataa-3′ (SEQ ID NO: 3) and primer 2: 5′-gcgaattctatatactgttgcaattctttctc-3′ (SEQ ID NO: 4). Here, the first denaturation was conducted once at 95° C. for 5 minutes and the second denaturation was conducted by repeating 30 cycles of holding at 95° C. for 50 seconds, annealing at 55° C. for 1 minute and extension at 72° C. for 1 minute and 30 seconds, and then the final extension was once conducted at 72° C. for 5 minutes. The cysK gene thus obtained was cut with the restriction enzyme EcoRI and the resulting segment was inserted into the plasmid p10499A (Park et al., FEMS Microbiol. Lett., 214:217-22, 2002) having the gntT104 promoter (Peekhaus and Conway, J. Bacteriol., 180:1777-85, 1998), which had been digested with the same restriction enzyme, to form the plasmid p104CysK. Then, the plasmid was cut with the restriction enzymes EcoRV and ScaI and cloned into the plasmid pACYC184 digested with the restriction enzyme EcoRV. The product was transformed into E. coli XL1-blue to prepare recombinant plasmid pAC104CysK (FIG. 2).

### Example 3

### Preparation of Recombinant Plasmid with IL-12p40 Gene Introduced

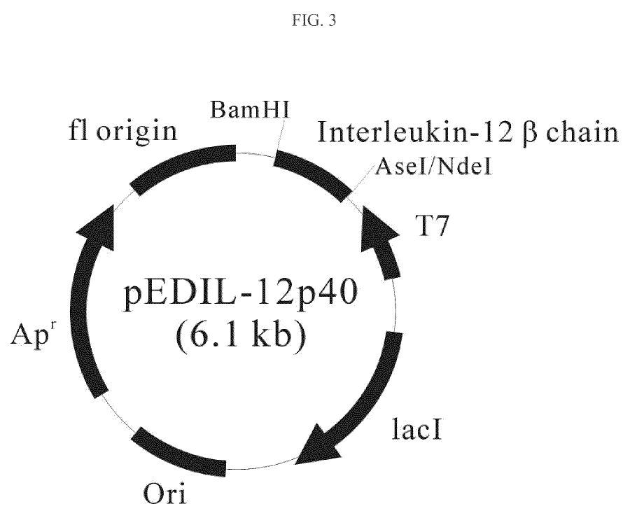

In order to express IL-112p40(interleukin 12 β chain) protein, the recombinant plasmid pEDIL-12p40 was prepared as follows. PCR was conducted using plasmid pUC18/p40 including human interleukin β chain gene as a template, and primer 3: 5′-ggctagcattaatgatatgggaactgaagaaagat-3′ (SEQ ID NO: 5) and primer 4: 5′-gccggatccttattaactgcagggcacaga-3′ (SEQ ID NO: 6) by following the same procedures as in Example 2 to obtain the IL-112p40 gene. The gene was digested with restriction enzymes AdeI and BamHI. The resulting segment was inserted into the leptin expression vector (Jeong and Lee, Appl. Environ. Microbiol., 65:3027-32, 1999), which had been digested with restriction enzymes NdeI and BamHI, to form plasmid pEDIL-12p40 (FIG. 3).

### Example 4

### Production of Human Leptin Protein by Coexpression System of CysK

The recombinant plasmid pAC 104CysK prepared in Example 2 and the conventional leptin expression plasmid pEDOb5 (Jeong and Lee, Appl. Environ. Microbiol. 65, 3027-32, 1999) were transformed simultaneously into E. coli BL21 (DE3) to prepare E. coli BL21(DE3)(pEDOb5)(pAC104CysK). This recombinant E. coli was cultured to produce leptin protein. Here, E. coli BL21 (DE3)(pEDOb5) transformed with only the conventional leptin expression plasmid pEDOb5 as a control was cultured under the same conditions to produce leptin protein.

Each of the transformed E. coli strains was inoculated into 10 mL of R/2 medium (KH₂PO₄ 6.75 g/L, (NH₄)₂HPO₄ 2 g/L, citric acid 0.85 g/L, trace metal solution (HCl 5M, FeSO₄.7H₂O 10 g/L, CaCl₂ 2 g/L, ZnSO₄.7H₂O 2.2 g/L, MnSO₄.5H₂O 0.54 g/L, CuSO₄.5H₂O 1 g/L, (NH₄)Mo₇O₂₄.4H₂O 0.1 g/L, Na₂B₄O₇.10H₂O 0.02 g/L), 5 mL/L, MgSO₄.7H₂O 0.7 g/L) with 10 g/L of glucose, cultured at 37° C. for overnight, transported to 200 mL of R/2 medium with 10 g/L of glucose and cultured at 37° C. for 8 hours. Then, 200 mL of the recombinant E. coli which had been cultured in the R/2 medium was inoculated into 1.8 L of R/2 medium with 10 g/L of glucose and cultured in an incubator kept at 37° C. and pH 6.88 while supplying a stock solution containing 700 g/L of glucose and 20 g/L of MgSO₄.7H₂O. Here, the stock solution was supplied according to changes of pH. For instance, when the pH of the medium was 6.88 or more, the stock solution was automatically adjusted and supplied at rate of 10 mL/min so that the glucose concentration in the fermentation chamber would be 0.7 g/L. Air and pure oxygen were automatically adjusted and supplied to maintain the dissolved oxygen (DO) in the medium at 40%. When the optical density (O.D.) of the culture broth as measured at 600 nm using a spectrophotometer was 30, 1 mM of IPTG(isopropyl-β-thiogalactoside) was added thereto to induce expression of leptin protein. In all the cultures, 100 mg/L of ampicillin and 30 mg/L of chloramphenicol were used to stabilize plasmids.

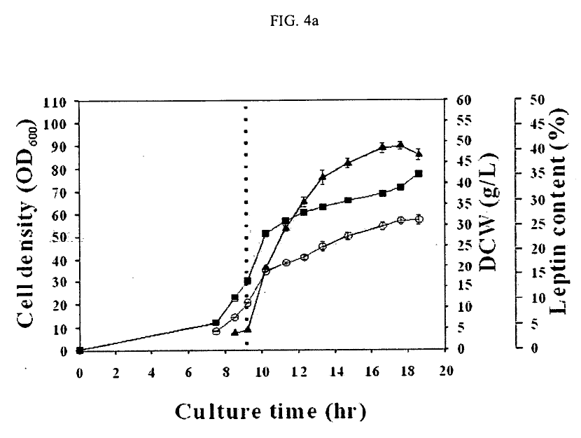

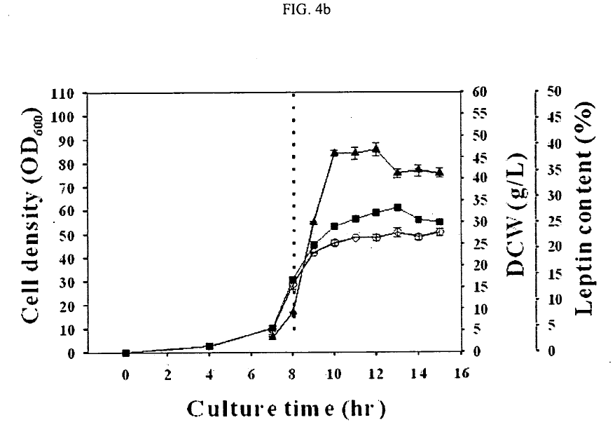

After inducing the expression of leptin protein, the culture broth was taken at every hour. Each aliquot was regularly diluted to be optical density (O.D.) of 5 and centrifuged at 6000 rpm for 5 minutes at 4° C. to form precipitates. The precipitates were suspended in 200 μl of TE buffer (Tris-HCl 10 mM, EDTA 1 mM) and subjected to 12% SDS-PAGE analysis according to a known method (FIG. 4a and FIG. 4b). FIG. 4a is a graph showing changes in cell density, dry cell weight and foreign protein quantity according to the culture time, when the control group is cultured and FIG. 4b is a graph showing changes in cell density, dry cell weight and foreign protein quantity according to the culture time, when the recombinant E. coli BL21 (DE3)(pEDOb5)(pAC104CysK) that can produce leptin is cultured, in which (▪) represents the optical density of cells, (o) represents the dry cell weight and (▴) represents the amount of prepared leptin. As shown in FIG. 4a, when the leptin expression plasmid was expressed alone, the expression of leptin reached the maximum after 8 hours from induction. From this fact, it was found that the production yield reached 0.457 g/L·h. On the other hand, as shown in FIG. 4b, when the leptin expression plasmid was coexpressed along with the pAC104CysK, the expression of leptin reached the maximum after 2 hours from induction. From this fact, it was found that the production yield reached 1.56 g/L·h.

Consequently, it was proved that the method for producing serine-rich protein by coexpression of the cysK gene according to the present invention could increase the leptin productivity by about 3.4 times as compared to the conventional method.

### Example 5

### Production of Serine-Rich Protein by cysK Coexpression System

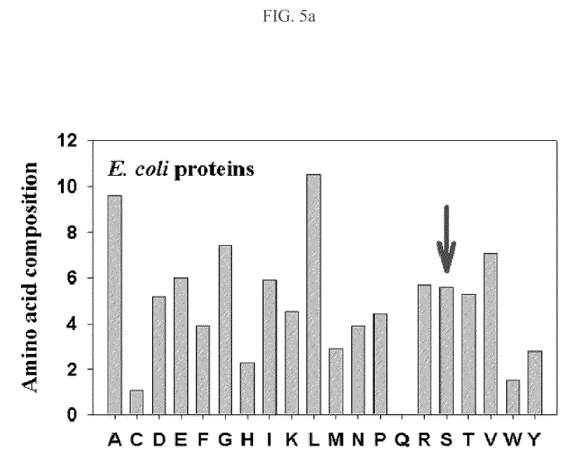

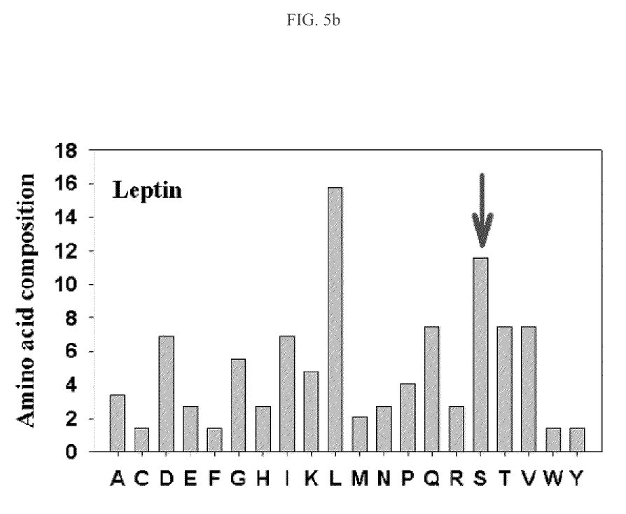

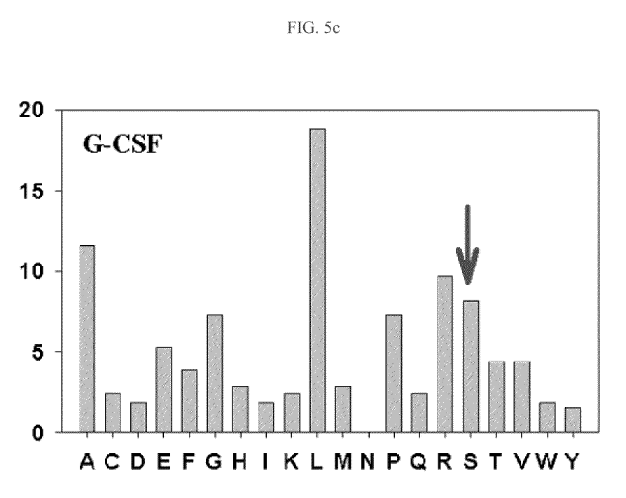

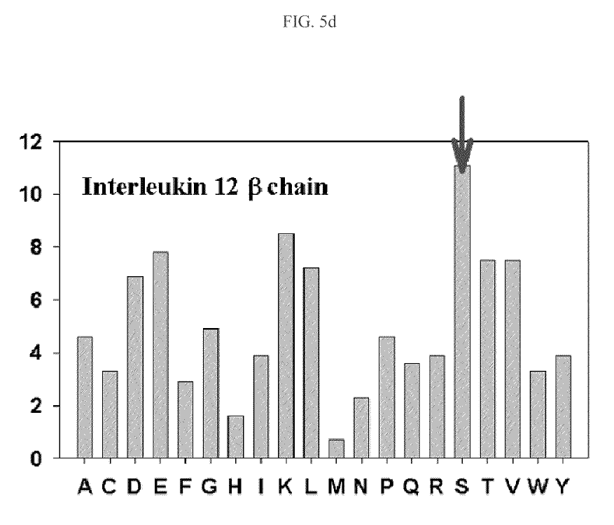

According to reports, leucine and alanine are prevalent in the average composition of amino acids of E. coli proteins (leucine 10.5% and alanine 9.6%) and serine is 5.6% on the average (FIG. 5a, FIG. 5b, FIG. 5c and FIG. 5d). FIGS. 5a to 5d are graphs showing compositional ratio of amino acids of proteins known up to date, in which FIG. 5a shows compositional ratio of amino acids of E. coli proteins, FIG. 5b shows compositional ratio of amino acids of leptin, FIG. 5c shows compositional ratio of amino acids of G-CSF and FIG. 5d shows compositional ratio of amino acids of IL-12p40.

As shown in FIG. 5b, the leptin protein, which is one of typical serine-rich proteins, comprises exceptionally much serine amino acid, in which the compositional ratio of serine is 11.6%. As shown in FIG. 5c, another known protein hG-CSF (human granulocyte-colony stimulating factor) has a leucine content of 19% and an alanine content of 12%, which are similar to proteins in E. coli, even though it has a serine content of 8.2%. As shown in FIG. 5d, another protein IL-12p40 which is also known as a serine-rich protein has a serine content of 11.1%.

In order to confirm if the results of Example 4 can be applied to production of all the serine-rich proteins, the present inventors subjected hG-CSF and the serine-rich protein IL-12p40 to the same method to produce proteins (Jeong and Lee, Protein Expr. Purif., 23:311-8, 2001).

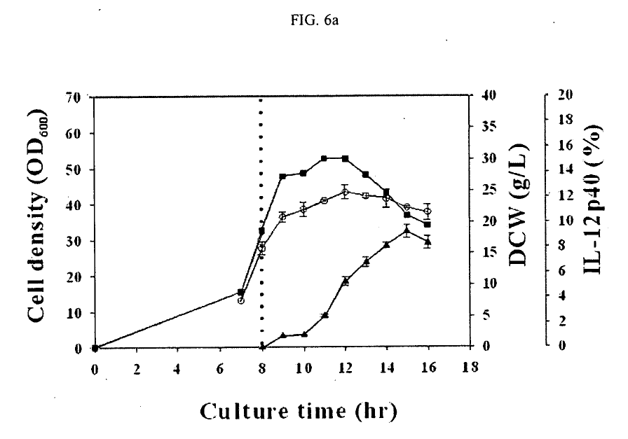

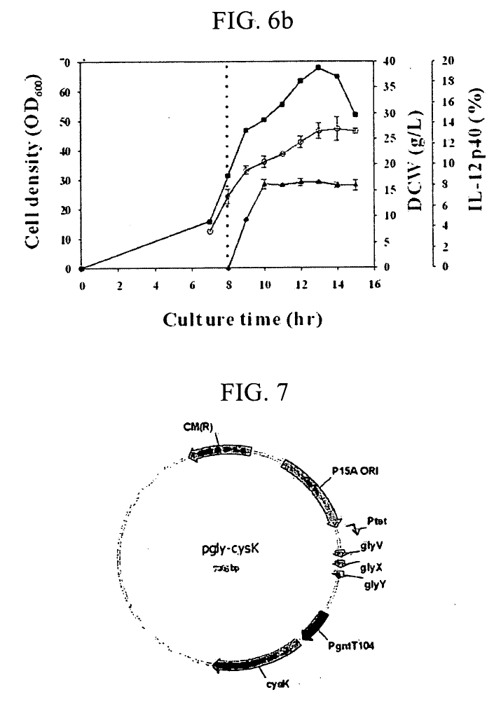

For the hG-CSF, the result was not similar to that of Example 4 because the protein production reached the peak in a short period of time (3 hours) without co-expression of the cysK gene. However, IL-12p40 showed increasing effects of productivity similar to leptin by coexpression of cysK gene (FIG. 6a and FIG. 6b).

FIG. 6a is a graph showing changes in cell density, dry cell weight and foreign protein quantity according to the culture time, when the IL-12p40-producing recombinant E. coli, BL21(DE3)(pEDIL-12p40), is cultured and FIG. 6b is a graph showing changes in cell density, dry cell weight and foreign protein quantity according to the culture time, when the recombinant E. coli BL21 (DE3)(pEDIL-12p40)(pAC104CysK) that can produce IL-12p40 and coexpress the cysK gene, is cultured, in which (▪) represents the optical density of cells, (o) represents the dry cell weight and (▴) represents the amount of prepared interleukin 12 β chain. As shown in FIG. 6a, when IL-112p40 was produced according to the method of Example 4 except for using pEDIL-12p40 prepared in Example 3, the expression of IL-12p40 reached the peak after 7 hours from induction. From this fact, it was found that the production yield was 0.090 g/L·h. On the other hand, as shown in FIG. 6b, when IL-12p40 was produced according to Example 4 except for using pAC104CysK prepared in Example 2 and pEDIL-12p40 prepared in Example 3, the expression reached the peak after 2 hours from induction, at the maximum yield of 0.349 g/L·h.

Consequently, it was proved that the method for producing serine-rich protein by coexpression of the cysK gene according to the present invention could increase the IL-12p40 productivity by about 3.9 times as compared to the conventional method.

### Example 6

### Production of Silk Protein by cysK Coexpression System

6-1: Construction of Recombinant Plasmid PSH32

All genetic manipulation procedures were carried out according to standard methods (Sambrook et al., Molecular cloning: a laboratory manual, 2nd Ed., Cold Spring Harbor Laboratory Press, Cold Spring Harbor, N.Y., 1989). To construct the recombinant plasmid pSH32, the plasmid pSH16a (Lee et al., Theories and Applications of Chem. Eng., 8(2):3969, 2002) was digested with the restriction enzymes SpeI and NheI (New England Biolabs, USA) to obtain a 1.7-kb fragment. The fragment was ligated with the plasmid pSH16a digested with the restriction enzyme SpeI, thus obtaining the recombinant plasmid pSH32.

6-2: Construction of Recombinant Plasmid pTet-glyVXY

All genetic manipulation procedures were carried out according to standard methods (Sambrook et al., Molecular cloning: a laboratory manual, 2nd Ed., Cold Spring Harbor Laboratory Press, Cold Spring Harbor, N.Y., 1989). PCR was performed using an E. coli W3110 strain (derived from E. coli K-12, λ⁻, F⁻, prototrophic) chromosome as a template and primers of SEQ ID NO: 7 and SEQ ID NO: 8.

SEQ ID NO 7:
5′-GCTCGATATCTAACGACGCAGAAATGCGAAA-3′;
SEQ ID NO 8:
5′-CATTGGATCCTAAGATTACAGCCTGAGGCTGTG-3′

The PCR reaction was performed using Pfu polymerase (SolGent, Korea) in the following conditions: first denaturation at 95° C. for 4 min, followed by 10 cycles of second denaturation at 95° C. for 20 sec, annealing at 51° C. for 30 sec and extension at 72° C. for 60 seconds, 19 cycles of denaturation at 95° C. for 20 sec, annealing at 60° C. for 30 sec and extension at 72° C. for 60 sec, and then final extension at 72° C. for 5 min.

The DNA fragment obtained by the PCR reaction was electrophoresed on agarose gel, thus obtaining a purified 479-bp PCR product. The PCR product was digested with the restriction enzymes BamHI and EcoRV (New England Biolabs, USA), and in order to use the promoter of a tetracycline-resistant gene (tet) which can be continually expressed, the plasmid pACYC 184 (New England Biolabs, USA) was also digested with the same restriction enzymes. The digested PCR product and plasmid were ligated with each other by T4 DNA ligase (Roche, Germany), and the ligated product was transformed into E. coli Top10 (F⁻ mcrA Δ(mrr-hsdRMS-mcrBC)C0lacZΔM15 ΔlacX74 recA1 araD139 Δ(ara-leu) 7697 galU galK rpsL (Str^{R}) endA1 nupG). The transformed strain was selected on LB agar solid medium (10 g/L tryptone, 5 g/L yeast extract, 5 g/L NaCl, and 15 g/L agar) containing 34 mg/L chloramphenicol, thus constructing the recombinant plasmid pTet-glyVXY. The constructed recombinant plasmid was confirmed by digestion with restriction enzymes and base sequence analysis.

6-3: Construction of Recombinant Plasmid pgly-CysK

All genetic manipulation procedures were carried out according to standard methods (Sambrook et al., Molecular cloning: a laboratory manual, 2nd Ed., Cold Spring Harbor Laboratory Press, Cold Spring Harbor, N.Y., 1989). The plasmid pTet-glyVXY prepared in Example 6-2 was digested with the restriction enzyme SalI (New England Biolabs, USA) and made blunt with Klenow enzyme. Then, the plasmid was ligated with the plasmid pAC104CysK (Han et al., Appl. Environ. Microbiol., 69(10):5772, 2003) digested with the restriction enzymes ClaI and EcoRV and were subjected to the same procedures as described in Example 6-2, thus obtaining the recombinant plasmid pgly-cysK (FIG. 7). The constructed recombinant plasmid was confirmed by digestion with restriction enzymes.

6-4: Expression of Silk Protein

In order to examine the effect of coexpression of a cysK gene and a silk protein, the plasmids pSH32 and pgly-cysK prepared in Examples 6-1 and 6-3, respectively, were transformed into an E. coli BL21 (DE3) strain (F-ompT hsdSB(rB-mB-) gal dcm (DE3) a prophage carrying the T7 RNA polymerase gene, New England Biolabs, USA). The transformed strain was cultured, and the expression of the silk protein in the strain was observed by SDS-PAGE. As control groups, the following strains were used: (i) an E. coli BL21 (DE3) strain transformed with the plasmids pACYC184 and pSH32, and (ii) an E. coli BL21 (DE3) strain transformed with the plasmids pSH32 and pTet-glyVXY which have been in Examples 6-1 and 6-2, respectively.

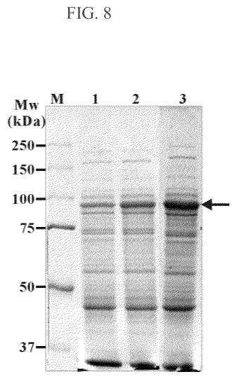

The transformed strains were inoculated into LB liquid media (containing 10 g/L tryptone, 5 g/L yeast extract, and 5 g/L NaCl) containing 34 mg/L chloramphenicol and 25 mg/L kanamycin and were cultured with continuous shaking at 30° C. at 180 rpm. When the optical density (O.D.) measured with a spectrophotometer at a wavelength of 600 nm after inoculation of each strain reached 0.4, 1 mM IPTG was added to each strain to induce the expression of the silk protein. At 5 hours after induction of the expression of the silk protein, the cultures were harvested. For recombinant protein analysis, each of the harvested cultures was centrifuged at 4° C. at 10,000 g for 10 minutes to obtain cell pellets which were then dissolved in TE buffer and 5× Laemmli sample buffer. The same amount (0.024 mg) of samples were taken from the cultures using 10% SDS-PAGE and stained with Coomassie brilliant blue R250 (Bio-Rad, USA), followed by quantification with GS-710 Calibrated Imaging Densitometer (Bio-Rad, USA) (FIG. 8).

As a result, the expression of the silk protein was increased by about 1.5 times when the expression of the silk protein-encoding gene along with the glycine tRNA gene was carried out (lane 2 in FIG. 8), and about 2.3 times when the coexpression of the silk protein-encoding gene, the glycine tRNA gene and the cysK gene was carried out (lane 3 in FIG. 8), as compared to when the expression of only the silk protein-encoding gene was carried out (lane 1 in FIG. 8). Namely, it was found that the expression of the silk protein was increased by about 1.5 times when the glycine tRNA gene and the silk protein-encoding gene along with the cysK gene were coexpressed, as compared to when the glycine tRNA gene and the silk protein-encoding gene were coexpressed in the absence of the cysK gene. In addition, it was observed that the growth of the cells was also significantly improved by the expression of the cysK gene.

Accordingly, it has been found that the production yield of the serine-rich protein is increased when the cysK gene and the serine-rich protein are coexpressed.

As described above, a particular part of the present invention is explained in detail. However, it is apparent to those skilled in the art that such concrete description is only for preferred embodiments and the present invention is not limited thereto. For example, as a method for overexpressing cysteine synthase, introduction of the cysK gene into a foreign protein expression vector or fusion into a chromosome of a host cell may achieve the same effect if the expression amount of the cysK gene. Therefore, the actual scope of the present invention is defined by the attached claims and equivalents thereof.

### INDUSTRIAL APPLICABILITY

As described in detail and proven, the present invention provides a method for preparing a foreign protein comprising culturing a bacterium containing the cysK gene and a gene encoding the foreign protein. More particularly, the present invention provides a method for preparing a serine-rich protein comprising culturing either a bacterium transformed with an expression vector containing a gene of a serine-rich foreign protein and an expression vector containing the cysK gene, or a bacterium transformed with an expression vector containing both the cysK gene and a gene encoding a serine-rich foreign protein and isolating the foreign protein from the culture.

Therefore, the present invention is expected to be widely used to increase the production yield of a serine-rich foreign protein in a process of producing a serine-rich foreign protein using a recombinant E. coli.

What is claimed is:

1. A process for preparing a serine-rich protein comprising the steps of:
culturing a bacterium containing cysK gene and a gene encoding serine-rich protein in a culture medium, thereby producing the serine-rich protein; and harvesting the serine-rich protein from the culture,
wherein the serine-rich protein has a cysteine content equal to or lower than the average cysteine content of host cell proteins of the bacterium; and
wherein said bacterium is a bacterium transformed with a vector containing the cysK gene and a vector containing the gene encoding the serine-rich protein.

2. A process for preparing a serine-rich protein comprising the steps of:
culturing a bacterium containing cysK gene and a gene encoding serine-rich protein in a culture medium, thereby producing the serine-rich protein; and harvesting the serine-rich protein from the culture,
wherein the serine-rich protein has a cysteine content equal to or lower than the average cysteine content of host cell proteins of the bacterium; and
wherein said bacterium is a bacterium transformed with a vector containing both the cysK gene and the gene encoding the serine-rich protein.

3. The process according to claim 1, wherein the cysK gene is derived from E. coli.

4. The process according to claim 2, wherein the cysK gene is derived from E. coli.

5. The process according to claim 1, wherein the serine-rich protein is any one among leptin, silk protein and sericin.

6. The process according to claim 2, wherein the serine-rich protein is any one among leptin, silk protein and sericin.

7. A bacterium transformed with a recombinant vector comprising both a cysK gene and a gene encoding a serine-rich protein, wherein the serine-rich protein has a cysteine content equal to or lower than the average cysteine content of host cell proteins of the bacterium.

8. The bacterium according to the claim 7, the serine-rich protein is any one among leptin, silk protein and sericin.

9. A bacterium transformed with a vector containing a cysK gene and a vector containing a gene encoding a serine-rich protein.

10. The bacterium according to the claim 9, the serine-rich protein is any one among leptin, silk protein and sericin.


SEQUENCE LISTING (8 sequences)

SEQ ID NO: 1 - protein, 34 aa, Artificial
  Synthetic construct of silk protein
SGRGGLGGTGAGMAAAAAMGGAGQGGYGGLGSQG

SEQ ID NO: 2 - protein, 38 aa, Artificial
  Synthetic construct of sericin
SSTGSSSNTDSNSNSVGSSTSGGSSTYGYSSNSRDGSV

SEQ ID NO: 3 - DNA, 31 bp, Artificial
  Synthetic construct of PCR primer
gcgaattcatgagtaagatttttgaagataa

SEQ ID NO: 4 - DNA, 32 bp, Artificial
  Synthetic construct of PCR primer
gcgaattctatatactgttgcaattctttctc

SEQ ID NO: 5 - DNA, 35 bp, Artificial
  Synthetic construct of PCR primer
ggctagcattaatgatatgggaactgaagaaagat

SEQ ID NO: 6 - DNA, 30 bp, Artificial
  Synthetic construct of PCR primer
gccggatccttattaactgcagggcacaga

SEQ ID NO: 7 - DNA, 31 bp, Artificial
  Synthetic construct of PCR primer
gctcgatatctaacgacgcagaaatgcgaaa

SEQ ID NO: 8 - DNA, 33 bp, Artificial
  Synthetic construct of PCR primer
cattggatcctaagattacagcctgaggctgtg